(19) United States
(12) Patent Application Publication
(10) Pub. No.: US 2022/0325216 A1
(43) Pub. Date: Oct. 13, 2022

(54) INTERMITTENT FEEDSTOCK TO GAS FERMENTATION

(71) Applicant: LanzaTech, Inc., Skokie, IL (US)

(72) Inventors: Robert John Conrado, Washington, DC (US); Sean Dennis Simpson, Evanston, IL (US); Michael Emerson Martin, Chicago, IL (US); Jason Carl Bromley, Chicago, IL (US); Richard Russell Rosin, Glencoe, IL (US); Darren Earl Youngs, Skokie, IL (US)

(21) Appl. No.: 17/658,575

(22) Filed: Apr. 8, 2022

Related U.S. Application Data
(60) Provisional application No. 63/173,258, filed on Apr. 9, 2021.
(60) Provisional application No. 63/173,247, filed on Apr. 9, 2021.

(51) Int. Cl. C12M 1/107; C12P 7/16; C12P 7/28; C12P 7/08; C12P 7/06; C12P 7/54; C12N 1/20

(52) U.S. Cl. CPC C12M 21/04; C12P 7/16; C12P 7/28; C12P 7/08; C12P 7/065; C12P 7/54; C12N 1/205; C12R 2001/145

(57) ABSTRACT

The disclosure provides methods to improve the economics of the gas fermentation process. A fermentation process is integrated with an industrial or syngas process and an reverse water gas shift process. An intermittent supply of reverse water gas shift process feedstock from the reverse water gas shift process is provided to the bioreactor for fermentation. The reverse water gas shift process feedstock may supplement or partially displace the C1 feedstock from the industrial or syngas process. Whether the reverse water gas shift process feedstock supplements or displaces the C1 feedstock may be based upon a function of the cost per unit of the C1 feedstock, the cost per unit of the reverse water gas shift process feedstock, and the value per unit of the fermentation product, or may depend upon the target gas ratio of the feedstock to the gas fermentation process.

### CROSS REFERENCE TO RELATED APPLICATIONS

This application claims the benefit of U.S. Provisional Patent Application Nos. 63/173,247 filed on Apr. 9, 2021, and 63/173,258 filed on Apr. 9, 2021, the entirety of which is incorporated herein by reference.

### FIELD

The invention relates to processes for improving the economics of a gas fermentation process. In particular, the invention relates to the combination of a gas fermentation process with an industrial or syngas process and a CO₂ to CO conversion process, such as a reverse water gas shift (rWGS) process. The CO₂ to CO conversion process may provide a feedstock that is continuously or intermittently passed to a bioreactor for gas fermentation in the fermentation process.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and/or hydrogen (H₂), into a variety of fuels and chemicals. Recently, however, gas fermentation has emerged as an alternative platform for the biological fixation of such gases. In particular, C1-fixing microorganisms have been demonstrated to convert gases containing CO₂, CO, CH₄, and/or H₂ into products such as ethanol and 2,3-butanediol.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, waste material may be recycled by pyrolysis, torrefaction, or gasification to generate syngas which is used as the substrate and/or C1-carbon source.

In certain embodiments, the industrial process is selected from fermentations, ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, oil extraction, or any combination thereof. Examples of specific processing steps within an industrial process include, catalyst regeneration, fluid catalyst cracking, catalyst regeneration. Air separation and direct air capture are other suitable industrial processes. In these embodiments, the substrate and/or C1-carbon source may be captured from the industrial process before it is emitted into the atmosphere, using any known method

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming . Examples of partial oxidation processes include thermal and catalytic partial oxidation processes, catalytic partial oxidation of natural gas, partial oxidation of hydrocarbons.

Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

With particular industrial or syngas processes the supply of gas may be insufficient for the fermentation process. When the supply of gas becomes insufficient for the fermentation process, the production rate of the fermentation process is less than optimal resulting in less products produced than what the fermentation process would otherwise be capable of producing.

Additionally, with a constantly adjusting market, the value of the products produced by the gas fermentation process varies. When the value of the products produced by the gas fermentation are high in comparison with the cost of producing such products, it is advantageous to increase the production rate of the fermentation process.

By increasing the production rate of the fermentation process at times when the market value of such products is high relative to the cost of producing such products, the economics of the fermentation process may be optimized.

Accordingly, there remains a need for improved integration of fermentation processes with industrial or syngas processes, where the problems associated with the supply of feedstock are curtailed and the fermentation process can produce at maximum levels at times when such production is economically optimal.

### BRIEF SUMMARY

Disclosed is a method of operating a fermentation process with a bioreactor containing a bacterial culture in a liquid nutrient medium, the method comprising passing a first C1 feedstock comprising one or both of CO and CO₂ from an industrial or syngas process to the bioreactor, wherein the first C1 feedstock has a cost per unit; passing a second C1 feedstock comprising CO₂ from an industrial or syngas process to at least one reverse water gas shift reactor of a reverse water gas shift process, wherein the reverse water gas shift process comprises a water electrolyzer and at least one water gas shift reactor, wherein the second C1 feedstock has a cost per unit, and wherein the second C1 feedstock and the first C1 feedstock are the same or different; optionally passing a hydrogen feedstock from the water electrolyzer to the reverse water gas shift reactor and generating a reverse water gas shift process feedstock comprising at least CO from the reverse water gas shift process wherein the reverse water gas shift process feedstock has a cost per unit; passing the reverse water gas shift process feedstock from the reverse water gas shift process to the bioreactor; and fermenting the culture to produce one or more fermentation products, wherein each of the one or more fermentation products has a value per unit. The hydrogen feedstock from the water electrolyzer may be passed directly to the bioreactor. The first C1 feedstock further comprises H₂. The second C1 feedstock may be generated through the direct capture of air and concentration of CO₂. The reverse water gas shift process feedstock may displace at least a portion of the first C1feedstock as a function of the cost per unit of the first C1 feedstock and the cost per unit of the reverse water gas shift feedstock. The reverse water gas shift process feedstock may displace at least a portion of the first C1 feedstock when the cost per unit of reverse water gas shift process feedstock is less than the cost per unit of first C1 feedstock. The reverse water gas shift process feedstock supplements the first C1 feedstock when the supply of the first C1 feedstock is insufficient for the fermentation process. The reverse water gas shift process feedstock supplements the first C1 feedstock as a function of the cost per unit of the reverse water gas shift process feedstock and the value per unit of the fermentation product. The reverse water gas shift process feedstock supplements the first C1 feedstock as a function of the cost per unit of the first C1 feedstock, the cost per unit of the reverse water gas shift process feedstock, and the value per unit of the fermentation product. The reverse water gas shift process feedstock supplements the first C1 feedstock when the cost per unit of the reverse water gas shift process feedstock is less than the value per unit of the fermentation product. The reverse water gas shift process feedstock further comprises unreacted H₂ and the amount of CO₂ fixed in the one or more fermentation products is increased as compared to the amount of CO₂ fixed in the absence of the unreacted H₂.

A method of operating a fermentation process with a bioreactor containing a bacterial culture in a liquid nutrient medium, the method comprising: passing a first C1 feedstock comprising one or both of CO and CO₂ from an industrial or syngas processes to the bioreactor, and fermenting the culture to produce one or more fermentation products, wherein the first C1 feedstock has a cost per unit and wherein each of the one or more fermentation products has a value per unit; intermittently operating a reverse water gas shift process which comprises at least one reverse water gas shift reactor and a water electrolyzer by passing a second C1 feedstock comprising CO₂ from an industrial or syngas process and optionally a H₂ stream generated by the water electrolyzer to the reverse water gas shift reactor to produce a reverse water gas shift feedstock comprising CO, any unreacted CO₂, and any unreacted H₂, wherein the second C1 feedstock and the first C1 feedstock are the same or different, and wherein the reverse water gas shift feedstock has cost per unit; passing the reverse water gas shift feedstock, when the reverse water gas shift process is operating, to the bioreactor to supplement the first C1 feedstock passed to the bioreactor, or to displace at least a portion of the first C1 feedstock passed to the bioreactor; operating the reverse water gas shift process during periods of time when i) the cost per unit of the reverse water gas shift feedstock is less than the cost per unit of the first C1 feedstock or ii) an increased quantity of fermentation product having a value per unit due the passing of the reverse water gas shift feedstock to the bioreactor provides a total value of fermentation product greater than a total cost of the first C1 feedstock and the reverse water gas shift feedstock based on the cost per unit of the first C1 feedstock and the cost per unit of the reverse water gas shift feedstock.

A method of controlling a fermentation process in a bioreactor containing a bacterial culture in a liquid nutrient medium, the method comprising: providing a first C1 feedstock comprising one or both of CO and CO₂ from an industrial or syngas processes, wherein the C1 feedstock has a H₂:CO₂ molar ratio and a H₂:CO molar ratio; intermittently operating a reverse water gas shift process which comprises at least one reverse water gas shift reactor and a water electrolyzer by passing a second C1 feedstock comprising CO₂ from an industrial or syngas process and optionally a H₂ stream generated by the water electrolyzer to the reverse water gas shift reactor to produce a reverse water gas shift feedstock comprising CO, any unreacted CO₂, and any unreacted H₂, wherein the second C1 feedstock and the first C1 feedstock are the same or different, and wherein the reverse water gas shift feedstock has a H_{2:}CO₂ molar ratio and a H₂:CO molar ratio; passing to the bioreactor i) the first C1 feedstock when the reverse water gas shift process is not operating, or ii) a combined feedstock comprising the C1 feedstock and the reverse water gas shift feedstock when the reverse water gas shift process is operating, or iii) either i) or ii) and the H₂ stream generated by the water electrolyzer, the combined feed having a H₂:CO₂ molar ratio and a H₂:CO molar ratio; fermenting, in the bioreactor, the culture using the first C1 feedstock and or the combined feedstock to produce one or more fermentation products, wherein the fermenting has a feedstock target range H₂:CO₂ molar ratio and a feedstock target range H₂:CO molar ratio; and operating the reverse water gas shift process during periods of time when the H₂:CO₂ molar ratio or the H₂:CO molar ratio of the C1 feedstock is not within the fermenting feedstock target range H₂:CO₂ molar ratio or feedstock target range H₂:CO molar ratio.

### BRIEF DESCRIPTION OF THE DRAWINGS

FIG. 1 is a schematic flow diagram depicting the integration of a fermentation process with an industrial or syngas process, and a CO₂ to CO conversion process shown as a reverse water gas shift process with a water electrolyzer.

FIG. 2 is a schematic flow diagram depicting the integration of a fermentation process with an industrial or syngas process, and a CO₂ to CO conversion process shown as a reverse water gas shift process with a water electrolyzer reverse water gas shift and further including a removal module for processing the C1 feedstock, in accordance with one embodiment.

FIG. 3 is a schematic flow diagram depicting the integration of a fermentation process with an industrial or syngas process, and a CO₂ to CO conversion process shown as a reverse water gas shift process with a water electrolyzer and further including a removal module for processing the reverse water gas shift feedstock, in accordance with one embodiment.

FIG. 4 is a schematic flow diagram depicting the integration of a fermentation process with an industrial or syngas process, and a CO₂ to CO conversion process shown as a reverse water gas shift process with a water electrolyzer and an optional pressure module for pressurizing the reverse water gas shift feedstock, and an optional pressure module for pressuring the C1 feedstock, in accordance with one embodiment.

FIG. 5 is a schematic flow diagram depicting the integration of a fermentation process with an industrial or syngas process, and a CO₂ to CO conversion process shown as a reverse water gas shift process with a water electrolyzer reverse water gas shift where a post-fermentation gaseous substrate is passed from the fermentation process to the reverse water gas shift process, in accordance with one embodiment.

FIG. 6 is a schematic flow diagram depicting integration of a fermentation process with an industrial or syngas process, and a CO₂ to CO conversion process shown as a reverse water gas shift process with a water electrolyzer and a removal module for processing the post-fermentation gaseous substrate, in accordance with one embodiment.

FIG. 7 is a schematic flow diagram depicting a fermentation process with an industrial or syngas process, and a CO₂ to CO conversion process shown as two reverse water gas shift processes with a water electrolyzer wherein at least one stream from the industrial or syngas process is combined with at least one stream from at least one of the two reverse water gas shift processes reverse water gas shift in accordance with one aspect of the disclosure.

FIG. 8 is a graph showing the price of electricity in Belgium over a period of nineteen days, with an average of one data point every four minutes.

### DETAILED DESCRIPTION

The disclosure provides a method for improving the performance and/or the economics of a fermentation process, the fermentation process defining a bioreactor containing a bacterial culture in a liquid nutrient medium, wherein the method comprises passing a C1 feedstock comprising one or both of CO and CO₂ from an industrial or syngas process to the bioreactor, wherein the C1 feedstock has a cost per unit, intermittently passing a reverse water gas shift feedstock comprising of CO from a reverse water gas shift process to the bioreactor, wherein the reverse water gas shift feedstock has a cost per unit, and fermenting the culture to produce one or more fermentation products, wherein each of the one or more fermentation products has a value per unit. In certain instances, a reverse water gas shift process and water electrolyzer process are both utilized in order to provide one or both of CO and H₂ to the bioreactor. The water electrolyzer process may be utilized in order to provide H₂ directly to the bioreactor

In certain instances, the C1 feedstock is derived from an industrial or syngas process selected from gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). In certain instances, the C1 feedstock is derived from a combination of two or more sources. In certain instances, the C1 feedstock may further comprise H₂.

The reverse water gas shift feedstock comprises CO, unreacted CO₂ and unreacted H₂, and water. The reverse water gas shift feedstock comprising CO is derived from the reverse water gas shift of a CO₂- and H₂-containing gaseous substrate. The CO₂- and H₂-containing gaseous substrate may be derived from any gas stream or streams containing CO₂ and H₂, in combination or separately. In particular instances, this CO₂- and H₂-containing gas stream(s) is derived at least in part from fermentation processes, from sugar based fermentation, from carbohydrate fermentation, from cellulosic based fermentation, from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). In particular instances, the CO₂-containing gaseous substrate is derived from a combination of two or more sources. H₂ may be produced by at least one of a water electrolyser, a hydrocarbon reforming source, a hydrogen purification source, a solid biomass gasification source, a solid waste gasification source, a coal gasification source, a hydrocarbon gasification source, a methane pyrolysis source, a refinery tail gas production source, or any combination thereof. Hydrogen produced may be supplied directly to the bioreactor.

In certain instances, the reverse water gas shift feedstock comprises H₂ which is unreacted in the reverse water gas shift reaction. The H₂ may have been passed to the reverse water gas shift process from a water electrolyzer or another H₂-generating processes. In an embodiment employing a water electrolyzer, water passed to the water electrolyzer may be obtained from numerous sources. For example, the water may be obtained from the industrial or syngas process and/or the fermentation process. In another embodiment, the water may be obtained from a wastewater treatment process. In other embodiments, the water is obtained from a combination of two or more sources.

In particular instances, the disclosure improves the economics of the fermentation process by displacing at least a portion of the C1 feedstock from the industrial or syngas process with reverse water gas shift feedstock from the reverse water gas shift process. In various instances when the reverse water gas shift feedstock comprises H₂, the reverse water gas shift feedstock either supplements or displaces at least a portion of the C1 feedstock from the industrial or syngas process as a means to adjust the molar ratio of H₂:CO or H₂:CO₂ of the feedstock being passed to the fermentation process. In certain instances, the reverse water gas shift feedstock comprising H₂ increases the molar ratio of H₂ in the feedstock being passed to the fermentation process.

The displacement of the C1 feedstock from the industrial or syngas process with reverse water gas shift feedstock from an reverse water gas shift process may be completed, at least in part, as a function of the cost per unit of the C1 feedstock and the cost per unit of the reverse water gas shift feedstock. In certain instances, the reverse water gas shift feedstock displaces at least a portion of the C1 feedstock when the cost per unit of reverse water gas shift feedstock is less than the cost per unit of C1 feedstock.

In particular instances, the disclosure improves the economics of the fermentation process by supplementing at least a portion of the C1 feedstock from the industrial or syngas process with reverse water gas shift feedstock from the reverse water gas shift process. The supplementing of the C1 feedstock with the reverse water gas shift feedstock may be completed, at least in part, when the supply of the C1 feedstock is insufficient for the fermentation process.

In certain instances, the reverse water gas shift feedstock supplements at least a portion of the C1 feedstock as a function of the cost per unit of the reverse water gas shift feedstock and the value per unit of the fermentation product.

In certain instances, the reverse water gas shift feedstock supplements at least a portion of the C1 feedstock as a function of the cost per unit of the C1 feedstock, the cost per unit of the reverse water gas shift feedstock, and the value per unit of the fermentation product.

In certain instances, the reverse water gas shift feedstock supplements the C1 feedstock when the cost per unit of the reverse water gas shift feedstock is less than the value per unit of the fermentation product. The cost per unit of reverse water gas shift feedstock may be less than the value per unit of the fermentation product when the cost of electricity is reduced. For example, during non-peak times of demand for electricity. Note however that the reverse water gas shift process is endothermic, and heat must be supplied, usually as utility steam which may factor into the cost per unit of reverse water gas shift feedstock. In certain instances, the cost of electricity is reduced due to the electricity being sourced from a renewable energy source. In certain instances, the renewable energy source is selected from solar, hydro, wind, geothermal, biomass, or combinations thereof. The reverse water gas shift reaction is endothermic and may require heat input from a source such as an electric source, a steam source, or from heat exchange with another exothermic process.

The supplementing of the C1 feedstock comprising CO₂ with reverse water gas shift feedstock comprising CO may result in a number of benefits, including but not limited to, increasing the amount of CO₂ fixed in the one or more fermentation products. Therefore, in various instances, reverse water gas shift feedstock comprising CO supplements the C1 feedstock comprising CO₂ to increase the amount of CO₂ fixed in the one or more fermentation products.

Intermittently operating the water electrolyzer and reverse water gas shift process may also provide a method of controlling a gas fermentation process in a bioreactor containing a bacterial culture in a liquid nutrient medium. The gas fermentation process has a feedstock target range H₂:CO₂ molar ratio and a feedstock target range H₂:CO molar ratio and operation within the target ranges provides increased efficiency of the gas fermentation process. The C1 feedstock comprising one or both of CO and CO₂ from an industrial or syngas processes has a H₂:CO₂ molar ratio and a H₂:CO molar ratio that may be within or outside of the target range for the particular fermentation process.

The H₂:CO₂ molar ratio and a H₂:CO molar ratio of C1 feedstock comprising one or both of CO and CO₂ from an industrial or syngas processes may be adjusted by combining with a reverse water gas shift feedstock comprising CO before introducing to the bioreactor. The relative ratios of the two feedstocks in the combined feedstock further provide a mechanism to adjust the H₂:CO₂ molar ratio and a H₂:CO molar ratio of the combined feedstock to reach the desired target ranges.

The reverse water gas shift process which comprises at least one reverse water gas shift reactor and a water electrolyzer may be operated intermittently to generate, when needed, the reverse water gas shift feedstock comprising at least CO to combine with the C1 feedstock when the C1 feedstock H₂:CO₂ molar ratio and a H₂:CO molar ratio not in the target desired range. A second C1 feedstock comprising CO₂ from an industrial or syngas process and a H₂ stream from the water electrolyzer or other source is passed to the reverse water gas shift reactor to produce a reverse water gas shift feedstock comprising CO and i) unreacted CO₂, ii) unreacted H₂ or both i) unreacted CO₂ and ii) unreacted H₂. The second C1 feedstock and the first C1 feedstock may be the same or different.

When the C1 feedstock comprising one or both of CO and CO₂ from an industrial or syngas processes has a H₂:CO₂ molar ratio and a H₂:CO molar ratio within the desired target range for the gas fermentation process, the reverse water gas shift process including the water electrolyzer need not be operated.

In particular embodiments, the C1 feedstock and or the reverse water gas shift feedstock contain proportions of various constituents that necessitate removal. In these instances, the C1 feedstock and or the reverse water gas shift feedstock are treated to remove one or more constituent prior to passing the C1 feedstock to the bioreactor. The constituents removed from the C1 feedstock may be: sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, tars, naphthalene, or combinations thereof. In particular embodiments, at least one constituent removed from the reverse water gas shift feedstock comprises methane. At least one of the constituents removed may be produced, introduced, and/or concentrated by the reverse water gas shift process. For example, methane may be produced, introduced, and/or concentrated by the reverse water gas shift of carbon dioxide and hydrogen. In various instances, methane is a by-product of the reverse water gas shift process. In particular embodiments, methane is produced and/or concentrated in the reverse water gas shift process.

In some embodiments, the C1 feedstock is passed to the fermentation process at pressure. In these embodiments, the C1 feedstock from the industrial or syngas process is passed to one or more pressure modules prior to being passed to the bioreactor for fermentation.

In some embodiments, the reverse water gas shift feedstock is passed to the fermentation process at pressure. In these embodiments, the reverse water gas shift feedstock from the reverse water gas shift process is passed to one or more pressure modules prior to being passed to the bioreactor for fermentation.

Additionally, the reverse water gas shift process may be conducted at pressure. When conducted at pressure, the feed(s) to the reverse water gas shift process are pressurized prior to being fed to the reverse water gas shift process. In certain instances, the material being reacted is a CO₂-and H₂-containing gas stream. In instances where the CO₂- and H₂-containing gas stream is pressurized prior to being reacted, the CO₂- and H₂-containing gas stream may be passed to a pressure module prior to being passed to the reverse water gas shift module.

In at least one embodiment, the method reduces the associated costs of producing various fermentation products. At least one of the one or more of the fermentation products may be ethanol, acetate, butyrate, 2,3-butanediol, lactate, butene, butadiene, ketones, methyl ethyl ketone, ethylene, acetone, isopropanol, lipids, 3-hydroypropionate, isoprene, fatty acids, 2-butanol, 1,2-propanediol, 1-propanol, and C6-C12 alcohols. At least one of the fermentation products may be further converted to at least one component of diesel, jet fuel, and/or gasoline.

At least one of the one or more fermentation products may be microbial biomass produced by the culture. At least a portion of the microbial biomass may be converted to a single cell protein (SCP). At least a portion of the single cell protein may be utilized as a component of animal feed.

The reverse water gas shift reaction is an endothermic reaction and heat may need to be introduced to the process. In at least one embodiment, heat may be generated , at least in part, by a renewable energy source. In certain instances, the renewable energy source is selected from solar, hydro, wind, geothermal, biomass, and combinations thereof. The renewable energy may provide the utilities needed to produce the heat. Steam may be generated to provide the heat, or burners may be used.

In certain embodiments, the industrial or syngas process may further produce a post-fermentation gaseous substrate. In various instances, this post-fermentation gaseous substrate comprises at least a portion of CO₂. In particular embodiments the post-fermentation gaseous substrate is passed to the reverse water gas shift process.

In particular instances, the post-fermentation gaseous substrate contains proportions of various constituents that necessitate removal. In these instances, the post-fermentation gaseous substrate is treated to remove one or more constituent prior to passing the post-fermentation gaseous substrate to the reverse water gas shift process. The constituents removed from the post-fermentation gaseous substrate may be selected from the group comprising: sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars, naphthalene, or combinations thereof.

In particular instances at least one constituent removed from the post-fermentation gaseous substrate comprises sulphur. At least one of these constituents removed may be produced, introduced, and/or concentrated by the fermentation process. For example, sulphur, in the form of hydrogen sulfide (H₂S) may be produced, introduced, and/or concentrated by the fermentation process. In particular embodiments, hydrogen sulfide is introduced in the fermentation process. In various embodiments, the post-fermentation gaseous substrate comprises at least a portion of hydrogen sulfide. Hydrogen sulfide may be a catalyst inhibitor or a catalyst poison, and may effect particular reverse water gas shift catalysts. In order to pass a non-inhibiting post-fermentation gaseous substrate to the reverse water gas shift process at least a portion of the hydrogen sulfide, or other constituent present in the post-fermentation gaseous substrate, may need to be removed by one or more removal modules.

In various embodiments, the constituent removed from the post-fermentation gaseous substrate, the industrial or syngas feedstock, and/or the reverse water gas shift feedstock is a microbe inhibitor and/or a catalyst inhibitor.

At least one removal module may be selected from hydrolysis module, acid gas removal module, deoxygenation module, catalytic hydrogenation module, particulate removal module, chloride removal module, tar removal module, and hydrogen cyanide removal module.

In embodiments wherein the reverse water gas shift process comprises a water electrolyzer, in addition to producing the H₂ enriched stream needed for the reverse water gas shift reaction, the water electrolyzer, also produces an O₂ enriched stream. At least a portion of the enriched stream may be passed to another process in need of an O₂ enriched stream. Utilizing the O₂ enriched stream in another process may further improve the performance and/or economics of the industrial or syngas process.

In various embodiments where the reverse water gas shift process reverse water gas shift feedstock comprises unreacted H₂, the H₂ may improve the fermentation substrate composition. Hydrogen provides energy required by the microorganism to convert carbon containing gases into useful products. When optimal concentrations of hydrogen are provided, the microbial culture can produce the desired fermentation products, for example ethanol, without the co-production of carbon dioxide.

The microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui.

The bacterial culture in the bioreactor may comprise a carboxydotrophic bacterium. The carboxydotrophic bacterium may be selected from Moorella, Clostridium, Ruminococcus, Acetobacterium, Eubacterium, Butyribacterium, Oxobacter, Methanosarcina, and Desulfotomaculum. The carboxydotrophic bacterium may be Clostridium autoethanogenum.

In one or more embodiments, the disclosure (i) decreases the cost associated with producing one or more fermentation product and/or (ii) increases the total amount of carbon converted to product, compared to a process without an integrated reverse water gas shift process.

The integration of a gas fermentation process with an industrial or syngas process and a CO₂ to CO conversion process, such as a reverse water gas shift process, results in many advantages. Furthermore, in the embodiment of this integration of a gas fermentation process with an industrial or syngas process and a CO₂ to CO conversion process, and particularly wherein the CO₂ to CO conversion process is a reverse water gas shift process, and where the CO₂ to CO conversion reverse water gas shift process intermittently supplies a feedstock, the integration is capable of substantially improving the performance and/or economics of the fermentation process. In another embodiment, the CO₂ to CO conversion process may continuously supplies a feedstock to the gas fermentation process.

The term “CO₂ to CO conversion system” as used herein refers to at least one unit selected from reverse water gas reaction system, thermo-catalytic conversion system, partial combustion system and plasma conversion system. A particular embodiment the CO₂ to CO conversion system is a reverse water gas shift reaction unit or system. For ease of understanding, the disclosure is discussed in terms of a reverse water gas shift process as the exemplary CO₂ to CO conversion system, however other CO₂ to CO conversion systems may be employed.

The term “reverse water gas reaction unit” or ““reverse water gas reaction process”” as used herein refers to a unit or system used for producing water from carbon dioxide and hydrogen, with carbon monoxide as a side product. The term “water gas” is defined as a fuel gas consisting mainly of carbon monoxide (CO) and hydrogen (H₂). The term ‘shift’ in water-gas shift means changing the water gas composition (CO:H₂) ratio. The ratio can be increased by adding CO₂ or reduced by adding steam to the reactor. The reverse water gas reaction unit may comprise a single stage or more than one stage. The different stages may be conducted at different temperatures and may use different catalysts.

The term “thermo-catalytic conversion”, another suitable CO₂ to CO conversion system, refers to a process to disrupt the stable atomic and molecular bonds of CO₂ and other reactants over a catalyst by using thermal energy as the driving force of the reaction to produce CO. Since CO₂ molecules are thermodynamically and chemically stable, if CO₂ is used as a single reactant, large amounts of energy are required. Therefore, often other substances such as hydrogen are used as a co-reactant to make the thermodynamic process easier. Many catalysts are known for the process such as metals and metal oxides as well as nano-sized catalyst metal-organic frameworks. Various carbon materials have been employed as carriers for the catalysts.

The term “partial combustion system” as used herein refers to a system where oxygen supplies at least a portion of the oxidant requirement for partial oxidation and the reactants carbon dioxide and water present therein are substantially converted to carbon monoxide and hydrogen.

The term “plasma conversion” refers to CO₂ conversion process, focusing on the combination of plasma with catalysts, called as plasma-catalysis. “Plasma” also called the “fourth state of matter,” is an ionized gas consisting of electrons, various types of ions, radicals, excited atoms, and molecules, besides neutral ground state molecules. The three most common plasma types for CO₂ conversion are: dielectric barrier discharges (DBDs), microwave (MW) plasmas, and gliding arc (GA) plasmas.

“Plasma conversion system” for CO₂ conversion comprises (i) high process versatility, allowing different kinds of reactions to be carried out (e.g., pure CO₂ splitting, as well as CO₂ conversion in the presence of a H-source, such as CH₄, H₂ or H₂O); (ii) low investment and operating costs; (iii) does not require the use of rare earth metals; (iv) a very modular setting, as plasma reactors scale up linearly with the plant output, allowing on-demand production; and (v) it can be very easily combined with various kinds of renewable electricity.

The terms “electrolysis module” and “electrolyzer” can be used interchangeably to refer to a unit that uses electricity to drive a non-spontaneous reaction. Electrolysis technologies are known in the art. Exemplary processes include alkaline water electrolysis, proton, or anion exchange membrane (PEM, AEM) electrolysis, and solid oxide electrolysis (SOE) (Ursua et al., Proceedings of the IEEE 100(2):410-426, 2012; Jhong et al., Current Opinion in Chemical Engineering 2:191-199, 2013). The term “faradaic efficiency” is a value that references the number of electrons flowing through an electrolyzer and being transferred to a reduced product rather than to an unrelated process. SOE modules operate at elevated temperatures. Below the thermoneutral voltage of an electrolysis module, an electrolysis reaction is endothermic. Above the thermoneutral voltage of an electrolysis module, an electrolysis reaction is exothermic. In some embodiments, an electrolysis module is operated without added pressure. In some embodiments, an electrolysis module is operated at a pressure of 5-10 bar.

A “CO₂ electrolysis module” refers to a unit capable of splitting CO₂ into CO and O₂ and is defined by the following stoichiometric reaction: 2CO₂+electricity→2CO+O₂. The use of different catalysts for CO₂ reduction impact the end product. Catalysts including, but not limited to, Au, Ag, Zn, Pd, and Ga catalysts, have been shown effective to produce CO from CO₂. In some embodiments, the pressure of a gas stream leaving a CO₂ electrolysis module is approximately 5-7 barg.

“H₂ electrolysis module,” “water electrolysis module,” and “H₂O electrolysis module” refer to a unit capable of splitting H₂O, in the form of steam, into H₂ and O₂ and is defined by the following stoichiometric reaction: 2H₂O+electricity→2H₂+O₂. An H₂O electrolysis module reduces protons to H₂ and oxidizes O²⁻ to O₂. H₂ produced by electrolysis can be blended with a C1-comprising gaseous substrate as a means to supply additional feedstock and to improve substrate composition.

H₂ and CO₂ electrolysis modules have 2 gas outlets. One side of the electrolysis module, the anode, comprises H₂ or CO (and other gases such as unreacted water vapor or unreacted CO₂). The second side, the cathode, comprises O₂ (and potentially other gases). The composition of a feedstock being passed to an electrolysis process may determine the presence of various components in a CO stream. For instance, the presence of inert components, such as CH₄ and/or N₂, in a feedstock may result in one or more of those components being present in the CO-enriched stream. Additionally, in some electrolyzers, O₂ produced at the cathode crosses over to the anode side where CO is generated and/or CO crosses over to the anode side, leading to cross contamination of the desired gas products.

The term “CO₂ to CO conversion process feedstock”, may include any effluent leaving the CO₂ to CO conversion process. In various instances, the CO₂ to CO conversion process feedstock is comprised of CO and H₂O, or combinations thereof. In certain instances, the CO₂ to CO conversion process feedstock may contain portions of unconverted H₂ and unconverted CO₂. The CO₂ to CO conversion process feedstock is the effluent from the CO₂ to CO conversion process, which is at least in part, introduced to the fermentation process.

The term “reverse water gas shift feedstock”, may include any effluent leaving the reverse water gas shift process. The reverse water gas shift feedstock is comprised of CO. In certain instances, the reverse water gas shift feedstock may contain portions of unconverted H₂ and unconverted CO₂. The reverse water gas shift feedstock is the effluent from the reverse water gas shift process, which is at least in part, introduced to the fermentation process.

The term “C1 feedstock”, may include any substrate leaving the industrial or syngas process comprising at least one single carbon gaseous compound. In various instances, the C1 feedstock is comprised of CO, H₂, CO₂, or combinations thereof. In some embodiment, the C1 feedstock may further comprise methane. Ultimately, a portion of the C1 feedstock from the industrial or syngas process is introduced to the fermentation process.

The terms “improving the economics”, “optimizing the economics” and the like, when used in relationship to a fermentation process, include, but are not limited to, the increase of the amount of one or more of the products produced by the fermentation process during periods of time in which the value of the products produced is high relative to the cost of producing such products. The economics of the fermentation process may be improved by way of increasing the supply of feedstock to the bioreactor, which may be achieved for instance by supplementing the C1 feedstock from the industrial or syngas process with CO₂ to CO conversion process feedstock such as reverse water gas shift feedstock from the reverse water gas shift process. The additional supply of feedstock may result in the increased efficiency of the fermentation process. Another means of improving the economics of the fermentation process is to select feedstock based upon the relative cost of the feedstock available. For example, when the cost of the C1 feedstock from the industrial or syngas process is higher than the cost of the CO₂ to CO conversion process feedstock, such as the reverse water gas shift feedstock from the reverse water gas shift process, the reverse water gas shift feedstock may be utilized to displace at least a portion of the C1 feedstock. By selecting feedstock based upon the cost of such feedstock the cost of producing the resulting fermentation product is reduced.

The CO₂ to CO conversion process such as the reverse water gas shift process is capable of supplying feedstock comprising CO. Because the reverse water gas shift process requires hydrogen, a water electrolyzer may be employed as the source of green hydrogen. In this situation, the cost of providing the reverse water gas shift feedstock in turn relies on the cost of the water electrolyzer. Therefore, “cost per unit of reverse water gas shift feedstock” may be expressed in terms of any given product produced by the fermentation process and any reverse water gas shift feedstock including the water electrolyzer, for example for the production of ethanol with the reverse water gas shift feedstock defined as CO and wherein the H₂ introduced to the reverse water gas shift process is provided by a water electrolyzer, the cost per unit of reverse water gas shift feedstock is defined by the following equation:

$$( \frac{\$ z}{MWh} ) \times ( \frac{1 MWh}{3.6 {GJ}_{electricity}} ) \times ( x \frac{{GJ}_{electricity}}{{GJ}_{H 2}} ) \times ( y \frac{{GJ}_{H 2}}{{GJ}_{ethanol}} )$$

where z represents the cost of power, x represents the reverse water gas shift efficiency which includes the water electrolyzer, and y represents the yield of ethanol.

In addition to the cost of feedstock, the fermentation process includes “production costs.” The “production costs” exclude the cost of the feedstock. “Production costs”, “marginal cost of production”, and the like, include the variable operating costs associated with running the fermentation process. This value may be dependent on the product being produced. The marginal cost of production may be represented by a fixed cost per unit of product, which may be represented in terms of the heating value of combustion of the product. For example, the calculation of the marginal cost of production for ethanol is defined by the following equation:

$$( \frac{\$ c}{metric ton} ) \times ( \frac{1 metric ton}{26.8 {GJ}_{ethanol}} )$$

where c represents the variable operating costs associated with running the bioreactor and 26.8 GJ represents the lower heating value of combustion of ethanol. In certain instances, the variable operating costs associated with running the bioreactor, c, is $200 for ethanol excluding the price of H₂CO/CO₂.

The fermentation process is capable of producing a number of products. Each product defining a different value. The “value of the product” may be determined based upon the current market price of the product and the heating value of combustion of the product. For example, the calculation for the value of ethanol is defined by the following equation:

$$( \frac{\$ z}{metric ton} ) \times ( \frac{1 metric ton}{26.8 {GJ}_{ethanol}} )$$

where z is the current value of ethanol per metric ton and 26.8 GJ represents the lower heating value of combustion of ethanol.

To optimize the economics of the fermentation process, the value of the product produced must exceed the “cost of producing” such product. The cost of producing a product is defined as the sum of the “cost of feedstock” and the “marginal cost of production.” The economics of the fermentation process may be expressed in terms of a ratio defined by the value of product produced compared to the cost of producing such product. The economics of the fermentation process is improved as the ratio of the value of the product compared to the cost of producing such product increases. The economics of the fermentation process may be dependent on the value of the product produced, which may change dependent, at least in part, on the fermentation process implemented, including but not limited to the bacterial culture and/or the composition of the gas used in the fermentation process. When ethanol is the product produced by the fermentation process the economics may be determined by the following ratio:

$$( \frac{\$ z}{{GJ}_{ethanol}} ) : ( \frac{\$ x}{{GJ}_{ethanol}} ) + ( \frac{\$ y}{{GJ}_{ethanol}} )$$

where z represents the value of ethanol, x represents the cost of feedstock, and y represents the marginal cost of production (excluding feedstock).

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation. In certain instances, the reverse water gas shift feedstock which includes a water electrolyzer increases the efficiency of the fermentation process.

The term “insufficient” and the like, when used in relation to the supply of feedstock for the fermentation process, includes, but is not limited to, lower than optimal amounts, whereby the fermentation process produces less quantity of fermentation product than the fermentation process otherwise would had the fermentation process been supplied with higher amounts of feedstock. For example, the supply of feedstock may become insufficient at times when the industrial or syngas process is not providing enough C1 feedstock to adequately supply the fermentation process. In an embodiment, the fermentation process is supplied with optimal amounts of feedstock such that the quantity of fermentation product is not limited by the feedstock supply.

“C1-containing gaseous substrate” may include any gas which contains one or both of carbon dioxide and carbon monoxide. The gaseous substrate will typically contain a significant proportion of CO₂, at least about 5% to about 100% CO₂ by volume. Additionally, the gaseous substrate may contain one or more of hydrogen (H₂), oxygen (O₂), nitrogen (N₂), and/or methane (CH₄).

While it is not necessary for the substrate to contain any hydrogen, the presence of H₂ should not be detrimental to product formation in accordance with methods of the disclosure. In particular embodiments, the presence of hydrogen results in an improved overall efficiency of alcohol production. In one embodiment, the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free.

The substrate may also contain some CO , such as about 1% to about 80% CO by volume, or 1% to about 30% CO by volume. In one embodiment, the substrate comprises less than or equal to about 20% CO by volume. In particular embodiments, the substrate comprises less than or equal to about 15% CO by volume, less than or equal to about 10% CO by volume, less than or equal to about 5% CO by volume or substantially no CO.

Substrate composition can be improved to provide a desired or optimum H₂:CO:CO₂ molar ratio. The desired H₂:CO:CO₂ molar ratio is dependent on the desired fermentation product of the fermentation process. For ethanol, the optimum H₂:CO:CO₂ molar ratio would be:

$$( x ) : ( y ) : ( \frac{x - 2 y}{3} ) ,$$

where x>2y, in order to satisfy the molar stoichiometry for ethanol production

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O .$$

Operating the fermentation process in the presence of hydrogen, has the added benefit of reducing the amount of CO₂ produced by the fermentation process. For example, a gaseous substrate comprising minimal H₂, will typically produce ethanol and CO₂ by the following molar stoichiometry [6CO+3H₂O→C₂H₅OH+4CO₂]. As the amount of hydrogen utilized by the C1 fixing bacterium increases, the amount of CO₂ produced decreases [i.e., 2CO+4H₂→C₂H₅OH+H₂O].

When CO is the sole carbon and energy source for ethanol production, a portion of the carbon is lost to CO₂ as follows:

6CO+3H₂O→C₂H₅OH+4CO₂ (ΔG °=−224.90 kJ/mol ethanol)

As the amount of H₂ available in the substrate increases, the amount of CO₂ produced decreases. At a molar stoichiometric ratio of 1:2 (CO/H₂), CO₂ production is completely avoided.

5CO+1H₂+2H₂O→1C₂H₅OH+3CO₂ (ΔG °=−204.80 kJ/mol ethanol)

4CO+2H₂+1H₂O→1C₂H₅OH+2CO₂ (ΔG °=−184.70 kJ/mol ethanol)

3CO+3H₂→1C₂H₅OH+1CO₂ (ΔG °=−164.60 kJ/mol ethanol)

“Gas stream” refers to any stream of substrate which is capable of being passed, for example, from one module to another, from one module to a bioreactor, from one process to another process, and/or from one module to a carbon capture means.

“Reactants” as used herein refer to a substance that takes part in and undergoes change during a chemical reaction. In particular embodiments, the reactants include, but are not limited to, CO₂, CO and/or H₂.

“Microbe inhibitors” as used herein refer to one or more constituent that slows down or prevents a particular chemical reaction or other process including the microbe. In particular embodiments, the microbe inhibitors include, but are not limited to, Oxygen (O₂), hydrogen cyanide (HCN), acetylene (C₂H₂), and BTEX (benzene, toluene, ethyl benzene, xylene).

“Catalyst inhibitor”, “adsorbent inhibitor”, and the like, as used herein, refer to one or more substance that decreases the rate of, or prevents, a chemical reaction. In particular embodiments, the catalyst and/or adsorbent inhibitors may include, but are not limited to, hydrogen sulfide (H₂S) and carbonyl sulfide (COS).

“Removal module”, “clean-up module”, “processing module” and the like includes technologies that are capable of either converting and/or removing microbe inhibitors, and/or catalyst inhibitors from the gas stream.

The term “constituents”, “contaminants”, and the like, as used herein, refers to the microbe inhibitors, and/or catalyst inhibitors that may be found in the gas stream. In particular embodiments, the constituents include, but are not limited to, sulphur-comprising compounds, aromatic compounds, alkynes, alkenes, alkanes, olefins, nitrogen-comprising compounds, phosphorous-comprising compounds, particulate matter, solids, oxygen, oxygenates, halogenated compounds, silicon-comprising containing compounds, carbonyls, metals, alcohols, esters, ketones, peroxides, aldehydes, ethers, tars, naphthalene, or combinations thereof. In an embodiment, the constituents removed by the removal module does not include carbon dioxide (CO₂).

The term “treated gas” refers to the gas stream that has been passed through at least one removal module and has had one or more constituent removed and/or converted.

The term “carbon capture” as used herein refers to the sequestration of carbon compounds including CO₂ and/or CO from a stream comprising CO₂ and/or CO and either: converting the CO₂ and/or CO into products; or converting the CO₂ and/or CO into substances suitable for long term storage; or trapping the CO₂ and/or CO in substances suitable for long term storage; or a combination of these processes.

The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Bubble Column, Gas Lift Fermenter, Static Mixer, a circulated loop reactor, a membrane reactor, such as a Hollow Fibre Membrane Bioreactor (HFM BR) or other vessel or other device suitable for gas-liquid contact. The bioreactor may be adapted to receive a gaseous substrate comprising CO or CO₂ or H₂ or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

“Nutrient media” or “Nutrient medium” is used to describe bacterial growth media. Generally, this term refers to a media containing nutrients and other components appropriate for the growth of a microbial culture. The term “nutrient” includes any substance that may be utilised in a metabolic pathway of a microorganism. Exemplary nutrients include potassium, B vitamins, trace metals and amino acids.

The term “fermentation broth” or “broth” is intended to encompass the mixture of components including nutrient media and a culture or one or more microorganisms. It should be noted that the term microorganism and the term bacteria are used interchangeably throughout the document.

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “desired composition” is used to refer to the desired level and types of components in a substance, such as, for example, of a gas stream. More particularly, a gas is considered to have a “desired composition” if it contains a particular component (i.e. CO, H₂, and/or CO₂) and/or contains a particular component at a particular proportion and/or does not contain a particular component (i.e. a constituent harmful to the microorganisms) and/or does not contain a particular component at a particular proportion. More than one component may be considered when determining whether a gas stream has a desired composition.

Unless the context requires otherwise, the phrases “fermenting”, “fermentation process” or “fermentation reaction” and the like, as used herein, are intended to encompass both the growth phase and product biosynthesis phase of the gaseous substrate.

A “microorganism” is a microscopic organism, especially a bacterium, archaea, virus, or fungus. The microorganism of the disclosure is typically a bacterium. As used herein, recitation of “microorganism” should be taken to encompass “bacterium.”

A “parental microorganism” is a microorganism used to generate a microorganism of the disclosure. The parental microorganism may be a naturally occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In a preferred embodiment, the parental microorganism is Clostridium autoethanogenum LZ1561, which was deposited on Jun. 7, 2010 with Deutsche Sammlung von Mikroorganismen and Zellkulturen GmbH (DSMZ) located at InhoffenstraBe 7B, D-38124 Braunschweig, Germany on Jun. 7, 2010 under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (i.e., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the disclosure is derived from a parental microorganism. In one embodiment, the microorganism of the disclosure is derived from Clostridium autoethanogenum, Clostridium ljungdahlii, or Clostridium ragsdalei. In a preferred embodiment, the microorganism of the disclosure is derived from Clostridium autoethanogenum LZ1561, which is deposited under DSMZ accession number DSM23693.

“Wood-Ljungdahl” refers to the Wood-Ljungdahl pathway of carbon fixation as described, i.e., by Ragsdale, Biochim Biophys Acta, 1784: 1873-1898, 2008. “Wood-Ljungdahl microorganisms” refers, predictably, to microorganisms containing the Wood-Ljungdahl pathway. Generally, the microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (i.e., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. In an embodiment, the C1-carbon source comprises one or both of CO and CO₂. A “C1-fixing microorganism” is a microorganism that has the ability to produce one or more products from a C1-carbon source. Typically, the microorganism of the disclosure is a C1-fixing bacterium.

An “anaerobe” is a microorganism that does not require oxygen for growth. An anaerobe may react negatively or even die if oxygen is present above a certain threshold. However, some anaerobes are capable of tolerating low levels of oxygen (i.e., 0.000001-5 vol% oxygen). Typically, the microorganism of the gas fermentation process is an anaerobe.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. Typically, the microorganism of the gas fermentation is an acetogen.

An “ethanologen” is a microorganism that produces or is capable of producing ethanol. Typically, the microorganism of the gas fermentation is an ethanologen.

An “autotroph” is a microorganism capable of growing in the absence of organic carbon. Instead, autotrophs use inorganic carbon sources, such as CO and/or CO₂. Typically, the microorganism of the gas fermentation is an autotroph.

A “carboxydotroph” is a microorganism capable of utilizing CO as a sole source of carbon and energy. Typically, the microorganism of the gas fermentation is a carboxydotroph.

A “methanotroph” is a microorganism capable of utilizing methane as a sole source of carbon and energy. In certain embodiments, the microorganism of the gas fermentation is a methanotroph or is derived from a methanotroph. In other embodiments, the microorganism of the gas fermentation is not a methanotroph or is not derived from a methanotroph.

“Substrate” refers to a carbon and/or energy source for the microorganism of the disclosure. Typically, the substrate is gaseous and comprises a C1-carbon source, for example, CO, CO₂, and/or CH₄. In an embodiment, the substrate comprises a C1-carbon source of CO or CO+CO₂. The substrate may further comprise other non-carbon components, such as H₂, N₂, or electrons.

The term “co-substrate” refers to a substance that, while not necessarily being the primary energy and material source for product synthesis, can be utilised for product synthesis when added to another substrate, such as the primary substrate.

The C1-carbon source may be a waste gas obtained as a by-product of an industrial process or from another source, such as automobile exhaust fumes, biogas or landfill gas or from electrolysis. The C1-carbon source may be syngas generated by pyrolysis, torrefaction, or gasification. Waste material may be recycled by pyrolysis, torrefaction, or gasification to generate syngas which is used as the C1-carbon source.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, oil extraction, or any combination thereof. Examples of specific processing steps within an industrial process include, catalyst regeneration, fluid catalyst cracking, catalyst regeneration. Air separation is another suitable industrial process. In these embodiments, the C1-carbon source may be captured from the industrial process before it is emitted into the atmosphere, using any known method

The C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, black liquor gasification, gasification of municipal solid waste, gasification of municipal liquid waste, gasification of industrial solid waste, gasification of industrial liquid waste, gasification of sewerage, gasification of sludge from wastewater treatment. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons.

Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation is performed in the absence of carbohydrate substrates, such as sugar, starch, lignin, cellulose, or hemicellulose.

The microorganism of the disclosure may be cultured with the gas stream to produce one or more products. For instance, the microorganism of the disclosure may produce or may be engineered to produce ethanol (WO 2007/117157), acetate (WO 2007/117157), butanol (WO 2008/115080 and WO 2012/053905), butyrate (WO 2008/115080), 2,3-butanediol (WO 2009/151342 and WO 2016/094334), lactate (WO 2011/11210 3), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2014/0369152), chorismate-derived products (WO 2016/191625), 3-hydroxybutyrate (WO 2017/066498), and 1,3-butanediol (WO 2017/0066498). In addition to one or more target products, the microorganism of the disclosure may also produce ethanol, acetate, and/or 2,3-butanediol. In certain embodiments, microbial biomass itself may be considered a product. These products may be further converted to produce at least one component of diesel, jet fuel, and/or gasoline. Additionally, the microbial biomass may be further processed to produce a single cell protein (SCP).

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients. An “excipient” may refer to any substance that may be added to the microbial biomass to enhance or alter the form, properties, or nutritional content of the animal feed.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the gas fermentation process may be engineered to produce products at a certain selectivity or at a minimum selectivity. In one embodiment, a target product account for at least about 5 wt. %, 10 wt. %, 15 wt. %, 20 wt. %, 30 wt. %, 50 wt. %, 75 wt. %, or 90 wt. % of all fermentation products produced by the microorganism of the gas fermentation process. In one embodiment, the target product accounts for at least 10 wt. % of all fermentation products produced by the microorganism, such that the microorganism has a selectivity for the target product of at least 10 wt. %. In another embodiment, the target product accounts for at least 30 wt. % of all fermentation products produced by the microorganism, such that the microorganism has a selectivity for the target product of at least 30 wt. %. In one embodiment, the target product accounts for at least 90 wt. % of all fermentation products produced by the microorganisms, such that the microorganism has a selectivity for the target product of at least 90 wt. %.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. In an embodiment the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be performed at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, the fermentation may be typically operated at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. In an embodiment, separated microbial cells may be returned to the bioreactor. The cell-free permeate remaining after target products have been removed is may also be returned to the bioreactor. Additional nutrients (such as B vitamins) may be added to the cell-free permeate to replenish the medium before it is returned to the bioreactor.

Carbon monoxide and water, with a co-product of methane, can be produced by a reverse water gas shift process, defined by the following molar stoichiometric reactions: H₂+CO₂→CO+H₂O and CO₂+4H₂→CH₄+2H₂O. The carbon monoxide produced by reverse water gas shift process can be used as a feedstock for gas fermentation. Furthermore, it is considered that the produced CO can be used in addition to a feedstock from an industrial or syngas process, as a means to provide additional feedstock and/or improve the fermentation substrate composition.

A water electrolyzer may be used to produce the hydrogen needed for the reverse water gas shift reaction. The water electrolyzer also capable of producing hydrogen from water, defined by the following molar stoichiometric reaction: 2H₂O+electricity→2H₂+O₂. The hydrogen produced by electrolysis can be used as a reactant in the reverse water gas shift reaction. This hydrogen may be used as the sole source of hydrogen for the reverse water gas shift reaction or alongside hydrogen from an industrial or syngas process, or as a means to provide additional feedstock and/or improve the fermentation substrate composition.

In one embodiment, the CO₂ to CO conversion process feedstock, particularly when the feedstock is a reverse water gas shift feedstock which involves the use of a water electrolyzer, may be used only at times when economically advantageous. In certain instances, the feedstock from the reverse water gas shift process involving a water electrolyzer may increase the efficiency of the fermentation process by reducing the costs associated with production.

The CO₂-containing substrate utilized by the CO₂ to CO conversion process, such as the reverse water gas shift process, for producing carbon monoxide may be derived from a number of sources. The CO₂-containing gaseous substrate may be derived, at least in part, from any gas containing CO₂, selected from gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). Additionally, the substrate may be captured from the industrial or syngas process before it is emitted into the atmosphere, using any conventional method. Furthermore, the CO₂-containing substrate may be derived from a combination of two or more of the above-mentioned sources.

Similarly, the H₂ containing stream used by the CO₂ to CO conversion process, and particularly the reverse water gas shift process, for producing CO may be obtained from several different sources. For example, a water electrolyzer may be employed to generate hydrogen from water, defined by the following molar stoichiometric reaction: 2H₂O+electricity→2H₂ +O₂. The hydrogen produced by electrolysis of water may be used to provide hydrogen to the reverse water gas shift process. The hydrogen produced by electrolysis of water may also be used as a feedstock for gas fermentation. The hydrogen produced by the electrolysis of water may be used as both a feedstock for gas fermentation and as a feedstock to a reverse water gas sift process. Other sources of H₂ rich gas streams include processes such as steam reformation of hydrocarbons, and in particular steam reformation of natural gas, partial oxidation of coal or hydrocarbons, by-products from electrolytic cells used to produce chlorine, styrene production processes, dehydrogenation of paraffins and olefins, and from other various refinery and petrochemical processes.

Gas streams typically will not be a pure CO₂ or pure H₂ streams and will contain proportions of at least one other component. For instance, each source may have differing proportions of CO₂, CO, H₂, and various constituents. Primarily due to the various constituents, the gas stream(s) may be processed prior to being introduced to the bioreactor and/or the reverse water gas shift process. The processing of the gas stream includes the removal and/or conversion of various constituents that may be microbe inhibitors and/or catalyst inhibitors. In an embodiment, the catalyst inhibitors are removed and/or converted prior to being passed to the reverse water gas shift process, and the microbe inhibitors are removed and/or converted prior to being passed to the bioreactor.

Typical constituents found in the gas stream that may need to be removed and/or converted include, but are not limited to, sulphur-comprising compounds, aromatic compounds, alkynes, alkenes, alkanes, olefins, nitrogen-comprising compounds, phosphorous-comprising compounds, particulate matter, solids, oxygen, oxygenates, halogenated compounds, silicon-comprising compounds, carbonyls, metals, alcohols, esters, ketones, peroxides, aldehydes, ethers, and tars, naphthalene, or combinations thereof.

These constituents may be removed by conventional removal modules known in the art. These removal modules may be selected from hydrolysis module, acid gas removal module, deoxygenation module, catalytic hydrogenation module, particulate removal module, chloride removal module, tar removal module, and hydrogen cyanide removal module.

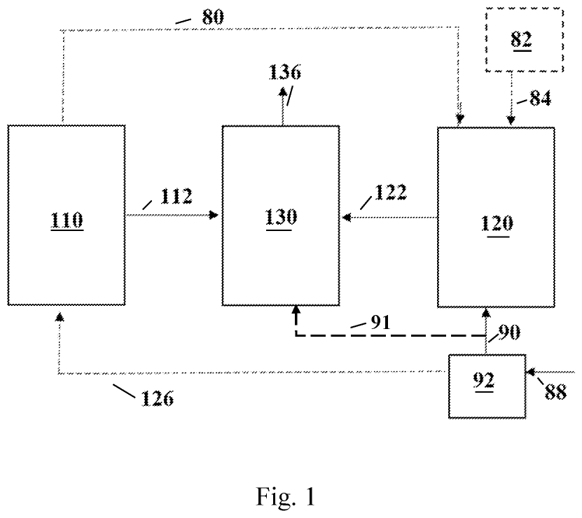

FIG. 1 shows the integration of an industrial or syngas process 110 and a reverse water gas shift process 120 with a fermentation process 130. The fermentation process 130 is capable of receiving C1 feedstock from the industrial or syngas process 110 and reverse water gas shift process feedstock from the reverse water gas shift process 120. The reverse water gas shift process feedstock from the reverse water gas shift process 120 may be fed to the fermentation process 130 continuously or intermittently. The C1 feedstock from the industrial or syngas process 110 is fed via a conduit 112 to the fermentation process 130, and the reverse water gas shift process feedstock from the reverse water gas shift process 120 is fed via a conduit 122 to the fermentation process 130. The reverse water gas shift process involved the reaction of CO₂ and H₂ to generate the reverse water gas shift process feedstock which comprises at least CO. The H₂ reactant needed for the reverse water gas shift reaction may be generated by water electrolyzer 92 which uses electricity to convert water in line 88 to H₂ in line 90 and O₂ in line 126. Hydrogen in line 90 is passed to reverse water gas shift process, and optionally, a portion of hydrogen from line 90 may bypass the reverse water gas shift process 120 and be directed by line 91 to gas fermentation process 130. The O₂ in line 126 may be passed for use in industrial or syngas gas process 110. The fermentation process 130 utilizes the reverse water gas shift process feedstock from the reverse water gas shift process 110 and the C1 feedstock from the industrial or syngas process 110 to produce one or more fermentation product 136. In some embodiments, second C1 feedstock from industrial or syngas process 110 is passed to reverse water gas shift process 120 in conduit 80. In another embodiment a second industrial or syngas process 82 may optionally provide a third C1 feedstock, comprising at least CO₂ to the reverse water gas shift process 120 via conduit 84. Second industrial or syngas process 82 may be different from industrial or syngas process 110. Second industrial or syngas process 82 may be a process for the separation of air.

In certain instances, the reverse water gas shift process feedstock comprises CO. In certain instances, the reverse water gas shift process feedstock comprises H₂. In certain instances, the reverse water gas shift process feedstock from the electrolyzer process 120 displaces at least a portion of the C1 feedstock from the industrial or syngas process 110. The reverse water gas shift process feedstock may displace at least a portion of the C1 feedstock as a function of the cost per unit of the C1 feedstock and the cost per unit of the reverse water gas shift process feedstock. In various instances, the reverse water gas shift process feedstock displaces at least a portion of the C1 feedstock when the cost per unit of reverse water gas shift process feedstock is less than the cost per unit of C1 feedstock.

The cost per unit of reverse water gas shift process feedstock may be less than the cost per unit of the C1 feedstock when the cost of electricity is reduced, such as during non-peak times of demand for electricity. In certain instances, the cost of electricity is reduced due to the electricity being sourced from a renewable energy source. In certain instances, the renewable energy source is selected from solar, hydro, wind, geothermal, biomass, and combinations thereof. As the reaction is endothermic, electricity may be consumed though the operation of the reverse water gas shift process due to heating requirements.

The reverse water gas shift process feedstock from the reverse water gas shift process 120 may supplement the C1 feedstock from the industrial or syngas process 110. The reverse water gas shift process feedstock may supplement the C1 feedstock when the supply of the C1 feedstock is insufficient for the fermentation process. In certain instances, the reverse water gas shift process feedstock supplements the C1 feedstock as a function of the cost per unit of the reverse water gas shift process feedstock and the value per unit of the fermentation product 136. In certain instances, the reverse water gas shift process feedstock supplements the C1 feedstock as a function of the cost per unit of the C1 feedstock, the cost per unit of the reverse water gas shift process feedstock, and the value per unit of the fermentation product 136. The reverse water gas shift process feedstock from the reverse water gas shift process 120 may supplement the C1 feedstock when the cost per unit of the reverse water gas shift process feedstock is less than the value per unit of the fermentation product 136. In various instances, the supplementing of the C1 feedstock comprising CO₂ with reverse water gas shift process feedstock comprising H₂ increases the amount of CO₂ fixed in the one or more fermentation product 136.

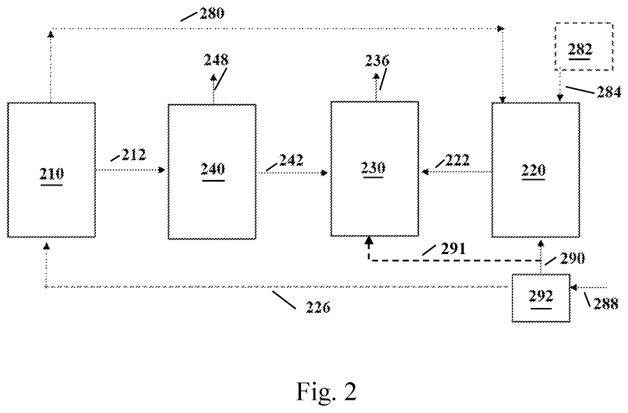

In particular instances, the C1 feedstock contains one or more constituent, and may require treatment prior to being sent to the fermentation process. FIG. 2 shows a removal module 240 for treating the C1 feedstock from the industrial or syngas process 210. When using a removal module 240, the C1 feedstock from the industrial or syngas process 210 is sent from the industrial or syngas process 210 to the removal module 240 via a conduit 212. The removal module 240 may remove and/or convert one or more constituent 248 in the C1 feedstock. The treated C1 feedstock is passed from the removal module 240 to the fermentation process 230 via a conduit 242.

In certain instances, the C1 feedstock is treated prior to being sent to the fermentation process, where the reverse water gas shift process feedstock from the reverse water gas shift process 220 is not treated prior to being sent to the fermentation process 230. When not being treated, the reverse water gas shift process feedstock may be sent via a conduit 222 from the reverse water gas shift process 220 to the fermentation process 230. The C1 feedstock from the industrial or syngas process 210 and the reverse water gas shift process feedstock from the reverse water gas shift process 220 may be used in the fermentation process 230 to produce one or more fermentation product 236. In some embodiments, second C1 feedstock from industrial or syngas process 210 is passed to reverse water gas shift process 220 in conduit 280. In another embodiment a second industrial or syngas process 282 may optionally provide a third C1 feedstock, comprising at least CO₂ to the reverse water gas shift process 220 via conduit 284. Second industrial or syngas process 282 may be different from industrial or syngas process 210. Second industrial or syngas process 282 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 292 which uses electricity to convert water in line 288 to H₂ in line 290 and O₂ in line 226. The O₂ in line 226 may be passed for use in industrial or syngas gas process 210. Hydrogen in line 290 is passed to reverse water gas shift process 220, and optionally, a portion of hydrogen from line 290 may bypass the reverse water gas shift process and be directed by line 291 to gas fermentation process 230. In particular instances, the reverse water gas shift process feedstock contains one or more constituent and may require treatment prior to being sent to the fermentation process.

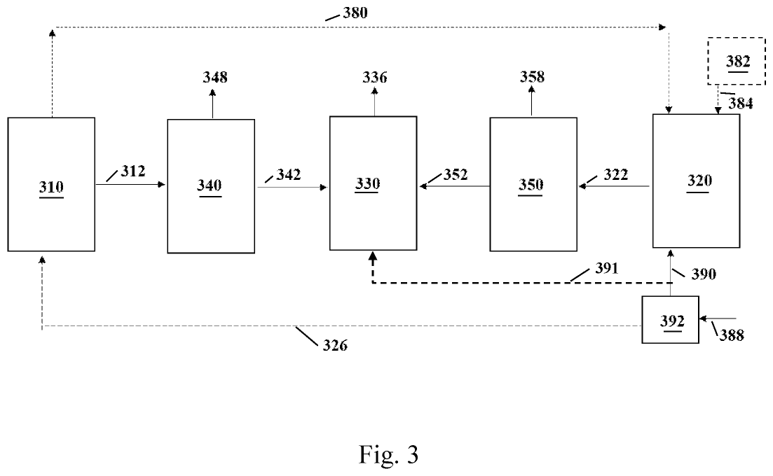

FIG. 3 shows a removal module 350 for treating the reverse water gas shift process feedstock from the reverse water gas shift process 320. When using a removal module 350, the reverse water gas shift process feedstock from the reverse water gas shift process 320 is sent from the reverse water gas shift process 320 to the removal module 350 via a conduit 322. The removal module 350 may remove and/or convert one or more constituent 358 in the reverse water gas shift process feedstock. The treated reverse water gas shift process feedstock is sent from the removal module 350 to the fermentation process 330 via a conduit 352.

In certain instances, both the C1 feedstock and the reverse water gas shift process feedstock are treated prior to being sent to the fermentation process. When treating the C1 feedstock, the C1 feedstock is sent from the industrial or syngas process 310 to the removal module 340 via a conduit 312 to remove and/or convert one or more constituent 348 in the C1 feedstock. The treated C1 feedstock is sent from the removal module 340 to the fermentation process 330 via a conduit 342. The C1 feedstock from the industrial or syngas process 310 and the reverse water gas shift process feedstock from the reverse water gas shift process 320 may be used in the fermentation process 330 to produce one or more fermentation product 336. In some embodiments, second C1 feedstock from industrial or syngas process 310 is passed to reverse water gas shift process 320 in conduit 380. In another embodiment a second industrial or syngas process 382 may optionally provide a third C1 feedstock, comprising at least CO₂ to the reverse water gas shift process 320 via conduit 384. Second industrial or syngas process 382 may be different from industrial or syngas process 310. Second industrial or syngas process 382 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 392 which uses electricity to convert water in line 388 to H₂ in line 390 and O₂ in line 326. The O₂ in line 326 may be passed for use in industrial or syngas gas process 310. Hydrogen in line 390 is passed to reverse water gas shift process 320, and optionally, a portion of hydrogen from line 390 may bypass the reverse water gas shift process 320 and be directed by line 391 to gas fermentation process 330.

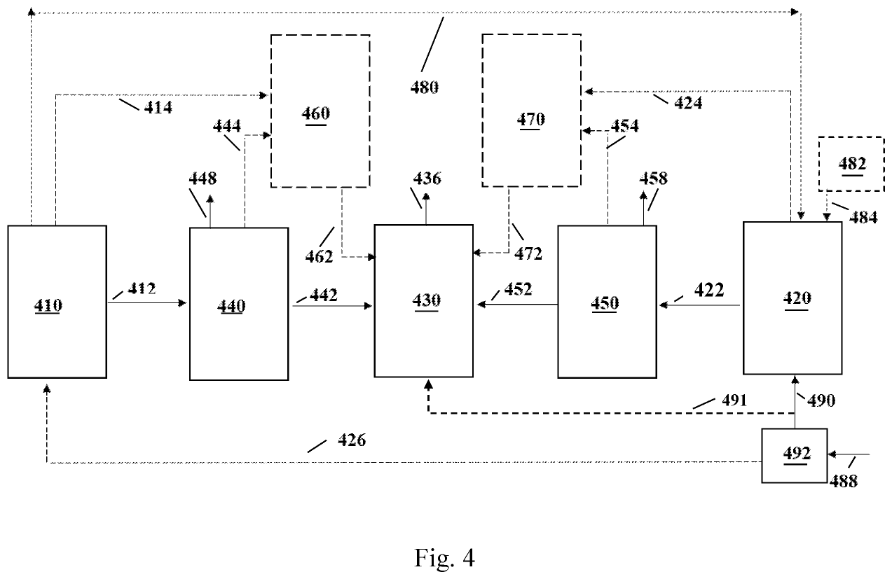

The feedstock may be pressurized prior to being passed to the fermentation process. FIG. 4 shows a pressure module 460 for pressurizing the C1 feedstock and a pressure module 470 for pressurizing the reverse water gas shift process feedstock. In certain instances, the C1 feedstock may be pressurized, while the reverse water gas shift process feedstock is not pressurized. In certain instances, the reverse water gas shift process feedstock may be pressurized, while the C1 feedstock is not pressurized. In various instances, the feedstock is pressurized without treatment. In various instances, the feedstock is pressurized following treatment. When pressurizing the C1 feedstock following treatment, the C1 feedstock is sent from the industrial or syngas process 410 to the removal module 440 via a conduit 412 to remove and/or convert one or more constituent 448. The treated C1 feedstock is sent from the removal module 440 to the pressure module 460 via a conduit 444. The pressurized C1 feedstock is sent from the pressure module 460 to the fermentation process 430 via a conduit 462. In instances where the C1 feedstock is not pressurized, the C1 feedstock may be sent from the removal module 440 to the fermentation process 430 via a conduit 442. In various instances where the C1 feedstock is pressurized without treatment, the C1 feedstock is sent from the industrial or syngas process 410 to the pressure module 460 via a conduit 414. When pressurizing the reverse water gas shift process feedstock following treatment, the reverse water gas shift process feedstock is sent from the reverse water gas shift process 420 to the removal module 450 via a conduit 422 to remove and/or convert one or more constituent 458. The treated reverse water gas shift process feedstock is sent from the removal module 450 to the pressure module 470 via a conduit 454. The pressurized reverse water gas shift process feedstock is sent from the pressure module 470 to the fermentation process 430 via a conduit 472. In instances where the reverse water gas shift process feedstock is not pressurized, the reverse water gas shift process feedstock may be sent from the removal module 450 to the fermentation process 430 via a conduit 452. In various instances where the reverse water gas shift process feedstock is pressurized without treatment, the reverse water gas shift process feedstock is sent from the reverse water gas shift process 420 to the pressure module 470 via a conduit 424. The C1 feedstock from the industrial or syngas process 410 and the reverse water gas shift process feedstock from the reverse water gas shift process 420 may be used in the fermentation process 430 to produce one or more fermentation product 436. In some embodiments, second C1 feedstock from industrial or syngas process 410 is passed to reverse water gas shift process 420 in conduit 480. In another embodiment a second industrial or syngas process 482 may optionally provide a third C1 feedstock, comprising at least CO₂ to the reverse water gas shift process 420 via conduit 484. Second industrial or syngas process 482 may be different from industrial or syngas process 410. Second industrial or syngas process 482 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 492 which uses electricity to convert water in line 488 to H₂ in line 490 and O₂ in line 426. The O₂ in line 426 may be passed for use in industrial or syngas gas process 410. Hydrogen in line 490 is passed to reverse water gas shift process 420, and optionally, a portion of hydrogen from line 490 may bypass the reverse water gas shift process 420 and be directed by line 491 to gas fermentation process 430.

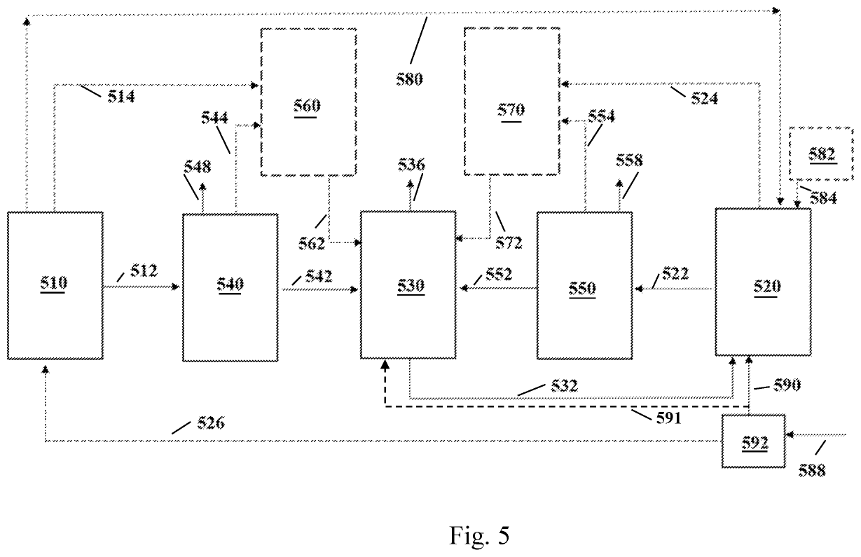

The fermentation process may produce a post-fermentation gaseous substrate in addition to the one or more fermentation product. This post-fermentation gaseous substrate may contain relatively high proportions of CO₂. In various instances, the post-fermentation gaseous substrate may be sent to the reverse water gas shift process. FIG. 5 shows the passing of a post-fermentation gaseous substrate from the fermentation process 530 to the electrolysis process 520 via a conduit 532. The fermentation process 530 may produce one or more fermentation product 536 and a post-fermentation gaseous substrate by utilizing feedstock from one or both of the industrial or syngas process 510 and/or the reverse water gas shift process 520. The C1 feedstock from the industrial or syngas process 510 may be pressurized by way of a pressure module 560. Pressurization may be completed with or without treatment. When pressurizing the C1 feedstock following treatment, the C1 feedstock is sent from the industrial or syngas process 510 to the removal module 540 via a conduit 512 to remove and/or convert one or more constituent 548. The treated C1 feedstock is sent from the removal module 540 to the pressure module 560 via a conduit 544. The pressurized C1 feedstock is sent from the pressure module 560 to the fermentation process 530 via a conduit 562. In instances where the C1 feedstock is not pressurized, the C1 feedstock may be sent from the removal module 540 to the fermentation process 530 via a conduit 542. In various instances where the C1 feedstock is pressurized without treatment, the C1 feedstock is sent from the industrial or syngas process 510 to the pressure module 560 via a conduit 514. When pressurizing the reverse water gas shift process feedstock following treatment, the reverse water gas shift process feedstock is sent from the reverse water gas shift process 520 to the removal module 550 via a conduit 522 to remove and/or convert one or more constituent 558. The treated reverse water gas shift process feedstock is sent from the removal module 550 to the pressure module 570 via a conduit 554. The pressurized reverse water gas shift process feedstock is sent from the pressure module 570 to the fermentation process 530 via a conduit 572. In instances where the reverse water gas shift process feedstock is not pressurized, the reverse water gas shift process feedstock may be sent from the removal module 550 to the fermentation process 530 via a conduit 552. In various instances where the reverse water gas shift process feedstock is pressurized without treatment, the reverse water gas shift process feedstock is sent from the reverse water gas shift process 520 to the pressure module 570 via a conduit 524. In some embodiments, second C1 feedstock from industrial or syngas process 510 is passed to reverse water gas shift process 520 in conduit 580. In another embodiment a second industrial or syngas process 582 may optionally provide a third C1 feedstock, comprising at least CO₂ to the reverse water gas shift process 520 via conduit 584. Second industrial or syngas process 582 may be different from industrial or syngas process 510. Second industrial or syngas process 582 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 592 which uses electricity to convert water in line 588 to H₂ in line 590 and O₂ in line 526. The O₂ in line 526 may be passed for use in industrial or syngas gas process 510. Hydrogen in line 590 is passed to reverse water gas shift process 520, and optionally, a portion of hydrogen from line 590 may bypass the reverse water gas shift process 520 and be directed by line 591 to gas fermentation process 530.

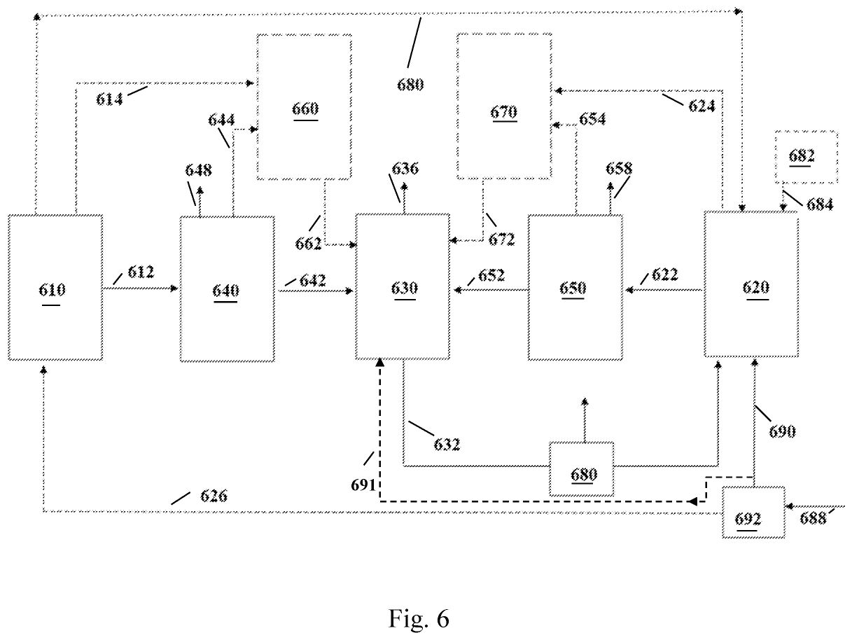

The post-fermentation gaseous substrate may contain one or more constituent that may need to be removed and/or converted prior to being passed to the reverse water gas shift process. FIG. 6 shows the passing of the post-fermentation gaseous substrate to a removal module 680 via a conduit 632 to remove and/or convert one or more constituent 688. The treated post-fermentation gaseous substrate is then passed from the removal module 680 to the reverse water gas shift process 620 via a conduit 682.

One or more of the constituents in the post-fermentation gaseous substrate may be produced, introduced, and/or concentrated by the fermentation process. In various embodiments, the one or more constituent produced, introduced, and/or concentrated by the fermentation step comprises sulphur. These constituents, including sulphur-comprising compounds, may decrease the efficiency of the reverse water gas shift process 620 if not removed and/or converted. The post-fermentation gaseous substrate may be treated so that it is suitable for electrolysis. By utilizing the post-fermentation gaseous substrate in the electrolysis module 620, an increased proportion of carbon may be captured by the process.

The fermentation process 630 may utilize the feedstock from one or both of the industrial or syngas process 610 an/or the reverse water gas shift process 620 to produce one or more fermentation product 636, where at least a portion of the reverse water gas shift process feedstock may be derived, at least in part, from the post-fermentation gaseous substrate. The C1 feedstock from the industrial or syngas process 610 may be pressurized by way of a pressure module 660. Pressurization may be completed with or without treatment. When pressurizing the C1 feedstock following treatment, the C1 feedstock is sent from the industrial or syngas process 610 to the removal module 640 via a conduit 612 to remove and/or convert one or more constituent 648. The treated C1 feedstock is sent from the removal module 640 to the pressure module 660 via a conduit 644. The pressurized C1 feedstock is sent from the pressure module 660 to the fermentation process 630 via a conduit 662. In instances where the C1 feedstock is not pressurized, the C1 feedstock may be sent from the removal module 640 to the fermentation process 630 via a conduit 642. In various instances where the C1 feedstock is pressurized without treatment, the C1 feedstock is sent from the industrial or syngas process 610 to the pressure module 660 via a conduit 614. When pressurizing the reverse water gas shift process feedstock following treatment, the reverse water gas shift process feedstock is sent from the reverse water gas shift process 620 to the removal module 650 via a conduit 622 to remove and/or convert one or more constituent 658. The treated reverse water gas shift process feedstock is sent from the removal module 650 to the pressure module 670 via a conduit 654. The pressurized reverse water gas shift process feedstock is sent from the pressure module 670 to the fermentation process 630 via a conduit 672. In instances where the reverse water gas shift process feedstock is not pressurized, the reverse water gas shift process feedstock may be sent from the removal module 650 to the fermentation process 630 via a conduit 652. In various instances where the reverse water gas shift process feedstock is pressurized without treatment, the reverse water gas shift process feedstock is sent from the reverse water gas shift process 620 to the pressure module 670 via a conduit 624. In some embodiments, second C1 feedstock from industrial or syngas process 610 is passed to reverse water gas shift process 620 in conduit 680. In another embodiment a second industrial or syngas process 682 may optionally provide a third C1 feedstock, comprising at least CO₂ to the reverse water gas shift process 620 via conduit 684. Second industrial or syngas process 682 may be different from industrial or syngas process 610. Second industrial or syngas process 682 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 692 which uses electricity to convert water in line 688 to H₂ in line 690 and O₂ in line 626. The O₂ in line 626 may be passed for use in industrial or syngas gas process 610. Hydrogen in line 690 is passed to reverse water gas shift process 620, and optionally, a portion of hydrogen from line 690 may bypass the reverse water gas shift process 620 and be directed by line 691 to gas fermentation process 630.

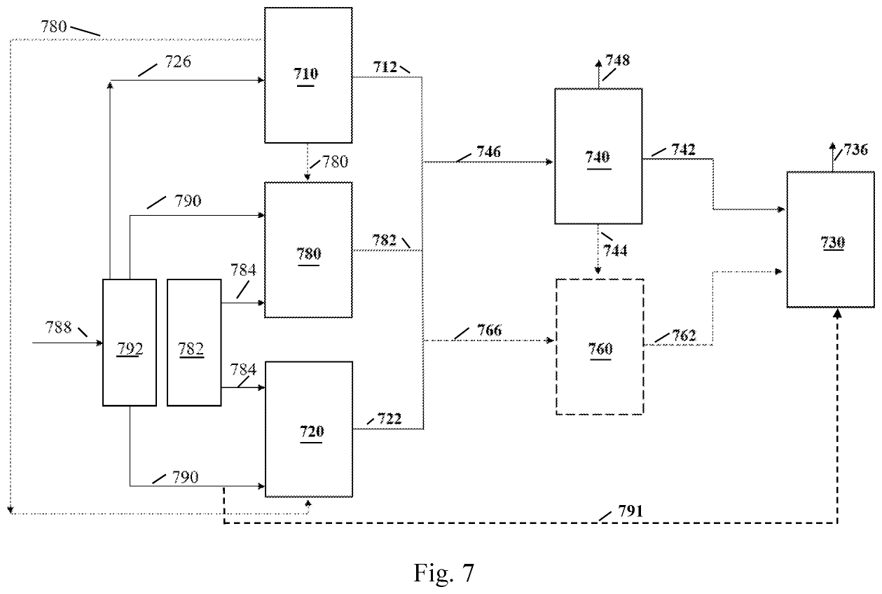

In various embodiments, the feedstock from one or more reverse water gas shift processes and the industrial or syngas process may be blended. FIG. 7 shows the blending of feedstock from the industrial or syngas process 710 and multiple reverse water gas shift processes 720, 780. The C1 feedstock from the industrial or syngas process 710 is sent via a conduit 712 to be blended. A first reverse water gas shift process feedstock from a first reverse water gas shift process 720 is sent via a conduit 722 to be blended. A second reverse water gas shift process feedstock from a second reverse water gas shift process 780 is sent via a conduit 782 to be blended. In certain instances, only the reverse water gas shift process feedstock from the first reverse water gas shift process 720 and the C1 feedstock from the industrial or syngas process 710 are blended. In certain instances, only the reverse water gas shift process feedstock from the second reverse water gas shift process 780 and the C1 feedstock from the industrial or syngas process 710 are blended. In certain instances, both the reverse water gas shift process feedstock from the first reverse water gas shift process 720 and the reverse water gas shift process feedstock from the second reverse water gas shift process 780 are blended. The blended feedstock may be sent via a conduit 746 to one or more removal module 740 to remove and/or convert one or more constituent 748.

The blended feedstock may be pressurized by way of a pressure module 760. Pressurization may be completed with or without treatment. When pressurizing the blended feedstock following treatment, the blended feedstock is sent via a conduit 746 to the removal module 740 to remove and/or convert one or more constituent 748. The treated blended feedstock is sent from the removal module 740 to the pressure module 760 via a conduit 744. The pressurized blended feedstock is sent from the pressure module 760 to the fermentation process 730 via a conduit 762 to produce one or more fermentation product 736. In instances where the blended feedstock is not pressurized, the blended feedstock may be sent from the removal module 740 to the fermentation process 730 via a conduit 742. In various instances where the blended feedstock is pressurized without treatment, the blended feedstock is sent via a conduit 766 to the pressure module 760.

In various instances the feedstock from one or more process may be intermittent while the other feedstock from one is more process is continuous. In certain instances, the reverse water gas shift process feedstock from one or more reverse water gas shift process 720, 780 are intermittent, while the C1 feedstock from the industrial or syngas process 710 is continuous. In certain instances, the C1 feedstock from the industrial or syngas process 710 is intermittent, while the reverse water gas shift process feedstock from one or more reverse water gas shift process 720, 780 are continuous. In certain instances, reverse water gas shift process feedstock from the first reverse water gas shift process 720 is intermittent, while the reverse water gas shift process feedstock from the second reverse water gas shift process 780 is continuous. In certain instances, the reverse water gas shift process feedstock from the second reverse water gas shift process 780 is intermittent, while the reverse water gas shift process feedstock from the first reverse water gas shift process 720 is continuous.

In some embodiments, second C1 feedstock from industrial or syngas process 710 is passed to reverse water gas shift processes 720 and or 780 in conduits 680. In another embodiment a second industrial or syngas process 782 may optionally provide a third C1 feedstock, comprising at least CO₂ to the reverse water gas shift processes 720 and or 780 via conduits 784. Second industrial or syngas process 782 may be different from industrial or syngas process 710. Second industrial or syngas process 782 may be a process for the separation of air. The reverse water gas shift process involves the reaction of CO₂ and H₂ to generate the reverse water gas shift process feedstock which comprises at least CO. The H₂ reactant needed for the reverse water gas shift reaction may be generated by water electrolyzer 792 which uses electricity to convert water in line 788 to H₂ in lines 790 and O₂ in line 726. The O₂ in line 726 may be passed for use in industrial or syngas gas process 710. Hydrogen in line 790 is passed to reverse water gas shift process 720, and optionally, a portion of hydrogen from line 790 may bypass the reverse water gas shift process 720 and be directed by line 791 to gas fermentation process 730.

In various embodiments, at least a portion of the reverse water gas shift process feedstock may be sent to storage. Certain industrial or syngas processes may include storage means for long-term or short-term storage of gaseous substrates and/or liquid substrates. In instances where at least a portion of the reverse water gas shift process feedstock is sent to storage, the reverse water gas shift process feedstock may be sent to the same storage means utilized by the industrial or syngas process, for example an existing gas holder at a steel mill. At least a portion of the reverse water gas shift process feedstock may be sent to independent storage means, where reverse water gas shift process feedstock is stored separately from the C1 feedstock from the industrial or syngas process. In certain instances, this stored feedstock from one or both of the industrial or syngas process and/or the one or more reverse water gas shift processes may be used by the fermentation process at a later time.

In various embodiments, the disclosure provides an integrated process wherein the utilities needed to provide heat to the endothermic reverse water gas shift process is provided, at least in part, from a renewable energy source. In certain instances, the renewable energy source is solar, hydro, wind, geothermal, biomass, or combinations thereof. Similarly, the water electrolyzer used to generate H₂ reactant for the reverse water gas shift reaction may be operated, at least in part, using a renewable energy source. More specifically, the electricity or electrons used in the water electrolyzer may be generated at least in part from a renewable energy source, such as those described above.

Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

In addition to increasing the efficiency of the fermentation process, the reverse water gas shift process may increase the efficiency of the industrial or syngas process. The increase in efficiency of the industrial or syngas process may be achieved through use of the water electrolyzer by-product, oxygen. Specifically, the O₂ by-product of the electrolyzer process may be used by the C1-generating industrial or syngas process. Many C1-generating industrial or syngas processes require O₂ for use in their processes. By utilizing the O₂ by-product from the water electrolyzer used in conjunction with the water gas shift process, the costs of producing O₂ specifically for the C1-generating industrial or syngas process can be reduced and/or eliminated. Passing the O₂ by-product from the electrolyzer process is exemplified in FIGS. 1-7 where the O₂ by-product is passed through a conduit, 126, 226, 326, 426, 526, 626, and 728 respectively, from the water electrolyzer to the industrial or syngas process. Similarly, methane may be generated in the reverse water gas shift process according to the Sabatier reaction and the methane generated may be separated and used in the industrial or syngas process.

Examples of C1-generating industrial or syngas processes which involve partial oxidation reactions, and thus require O₂ input, which could benefit from the additional integration involving the O₂ by-product from the water electrolyzer include industrial or syngas Basic Oxygen Furnace (BOF) reactions, COREX or FINEX steel making processes, Blast Furnace (BF) processes, ferroalloy production processes, titanium dioxide production processes, and gasification processes. Gasification processes include, but are not limited, to municipal solid waste gasification, biomass gasification, pet coke gasification and coal gasification. In one or more of these industrial or syngas processes, the O₂ from the water electrolyzer process may be used to off-set or completely replace the O₂ typically supplied through air separation. As to gasification processes, the O₂ by-product from the water electrolyzer may be used in an oxygen blown gasifier.

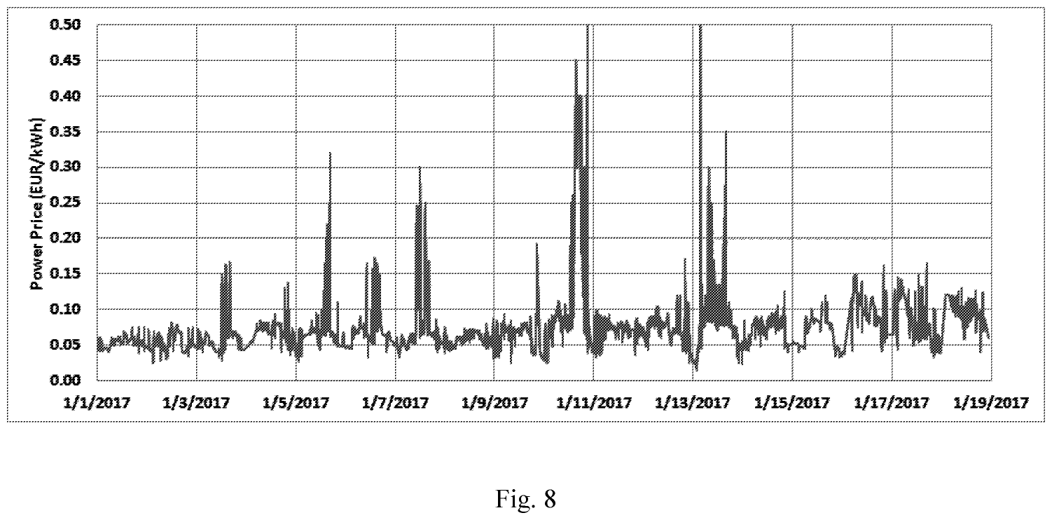

The need for the current disclosure is illustrated by FIG. 8, which depicts the price of electricity in Belgium over a nineteen-day period. FIG. 8 highlights the difference between the average price of electricity (roughly 0.05 EUR/kWh) and the minimum/maximum price of electricity over a period of time. Due to the vast difference in the price of electricity in a given location, and the effect of electricity price on the efficiency of reverse water gas shift process feedstock and its' use of a water electrolyzer, as a gas source for fermentation, it is largely advantageous to have a flexible approach for the utilization of the process. For example, utilizing a reverse water gas shift process which involves a water electrolyzer as a gas source for fermentation when electricity is relatively inexpensive, such as during off-peak electricity demand times, and discontinuing use for periods of time in which prices are high, such as during peak electricity demand times. This demand-responsive utilization of reverse water gas shift with water electrolysis can add tremendous value to a gas fermentation facility.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (i.e., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated. Unless otherwise indicated, ratios are molar ratios, and percentages are on a weight basis.

All methods described herein can be performed in any suitable order unless otherwise indicated herein or otherwise clearly contradicted by context. The use of any and all examples, or exemplary language (i.e., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of operating a fermentation process with a bioreactor containing a bacterial culture in a liquid nutrient medium, the method comprising:
a. passing a first C1 feedstock comprising one or both of CO and CO₂ from an industrial or syngas process to the bioreactor, wherein the first C1 feedstock has a cost per unit;
b. passing a second C1 feedstock comprising CO₂ from an industrial or syngas process to at least one reverse water gas shift reactor of a reverse water gas shift process, wherein the reverse water gas shift process comprises a water electrolyzer and at least one reverse water gas shift reactor, wherein the second C1 feedstock has a cost per unit, and wherein the second C1 feedstock and the first C1 feedstock are the same or different;
c. optionally passing a hydrogen feedstock from the water electrolyzer to the reverse water gas shift reactor and generating a reverse water gas shift process feedstock comprising at least CO from the reverse water gas shift process wherein the reverse water gas shift process feedstock has a cost per unit;
d. optionally passing a hydrogen feedstock from the water electrolyzer to the bioreactor wherein the hydrogen feedstock from the water electrolyzer has a cost per unit;
e. passing the reverse water gas shift process feedstock from the reverse water gas shift process to the bioreactor; and
f. fermenting the culture to produce one or more fermentation products, wherein each of the one or more fermentation products has a value per unit.

2. The method of claim 1, wherein the first C1 feedstock further comprises H₂.

3. The method of claim 1, wherein the second C1 feedstock is generated through carbon capture by concentration of CO₂ from air.

4. The method of claim 1, wherein the reverse water gas shift process feedstock displaces at least a portion of the first C1 feedstock as a function of the cost per unit of the first C1 feedstock and the cost per unit of the reverse water gas shift feedstock.

5. The method of claim 1, wherein the reverse water gas shift process feedstock displaces at least a portion of the first C1 feedstock when the cost per unit of reverse water gas shift process feedstock is less than the cost per unit of first C1 feedstock.

6. The method of claim 1, wherein the reverse water gas shift process feedstock supplements the first C1 feedstock when the supply of the first C1 feedstock is insufficient for the fermentation process.

7. The method of claim 1, wherein the reverse water gas shift process feedstock supplements the first C1 feedstock as a function of the cost per unit of the reverse water gas shift process feedstock and the value per unit of the fermentation product.

8. The method of claim 1, wherein the reverse water gas shift process feedstock supplements the first C1 feedstock as a function of the cost per unit of the first C1 feedstock, the cost per unit of the reverse water gas shift process feedstock, and the value per unit of the fermentation product.

9. The method of claim 1, wherein the reverse water gas shift process feedstock supplements the first C1 feedstock when the cost per unit of the reverse water gas shift process feedstock is less than the value per unit of the fermentation product.

10. The method of claim 9, wherein the reverse water gas shift process feedstock further comprises unreacted H₂ and the amount of CO₂ fixed in the one or more fermentation products is increased as compared to the amount of CO₂ fixed in the absence of the unreacted H₂.

11. The method of claim 1, wherein the first C1 feedstock, the second C1 feedstock, the reverse water gas shift process feedstock, or any combination thereof are treated to remove one or more constituent prior to passing to the bioreactor.

12. The method of claim 1, wherein the first C1 feedstock, the second C1 feedstock, the reverse water gas shift process feedstock, or any combination thereof are pressurized prior to passing the C1 feedstock to the bioreactor.

13. The method of claim 1, wherein at least one of the fermentation products is selected from ethanol, acetate, butyrate, 2,3-butanediol, lactate, butene, butadiene, ketones, methyl ethyl ketone, ethylene, acetone, isopropanol, lipids, 3-hydroypropionate, isoprene, fatty acids, 2-butanol, 1,2-propanediol, 1-propanol, microbial biomass or any combination thereof.

14. The method of claim 1, wherein the water electrolyzer is powered, at least in part, by a renewable energy source.

15. The method of claim 14, wherein the renewable energy source is selected from solar, hydro, wind, geothermal, biomass, nuclear, or combinations thereof.

16. The method of claim 1, wherein the culture further produces a post-fermentation gaseous substrate and the method further comprising passing the post-fermentation gaseous substrate to the reverse water gas shift process, industrial or syngas process, or both.

17. The method of claim 16, wherein the post-fermentation gaseous substrate is treated to remove one or more constituents prior to being passed to the reverse water gas shift process or industrial or syngas process.

18. The method of claim 16 wherein the water electrolyzer further generates an O₂ stream that is passed to a gasifier.

19. A method of operating a fermentation process with a bioreactor containing a bacterial culture in a liquid nutrient medium, the method comprising:
a. passing a first C1 feedstock comprising one or both of CO and CO₂ from an industrial or syngas processes to the bioreactor, and fermenting the culture to produce one or more fermentation products, wherein the first C1 feedstock has a cost per unit and wherein each of the one or more fermentation products has a value per unit;
b. intermittently operating a reverse water gas shift process which comprises at least one reverse water gas shift reactor and a water electrolyzer by passing a second C1 feedstock comprising CO₂ from an industrial or syngas process and optionally a H₂ stream from the water electrolyzer to the reverse water gas shift reactor to produce a reverse water gas shift feedstock comprising CO, any unreacted CO₂, and any unreacted H₂, wherein the second C1 feedstock and the first C1 feedstock are the same or different, and wherein the reverse water gas shift feedstock has cost per unit;
c. passing the reverse water gas shift feedstock, when the reverse water gas shift process is operating, to the bioreactor to supplement the first C1 feedstock passed to the bioreactor, or to displace at least a portion of the first C1 feedstock passed to the bioreactor; and
d. operating the reverse water gas shift process during periods of time when
i. the cost per unit of the reverse water gas shift feedstock is less than the cost per unit of the first C1 feedstock; or
ii. an increased quantity of fermentation product having a value per unit due the passing of the reverse water gas shift feedstock to the bioreactor provides a total value of fermentation product greater than a total cost of the first C1 feedstock and the reverse water gas shift feedstock based on the cost per unit of the first C1 feedstock and the cost per unit of the reverse water gas shift feedstock.

20. The method of claim 19 wherein intermittently operating a reverse water gas shift process is operating the reverse water gas shift process at full capacity.

21. The method of claim 19, wherein the first C1 feedstock, the second C1 feedstock, the second C1 feedstock, the reverse water gas shift feedstock, or any combination thereof are treated to remove one or more constituents prior to passing to the bioreactor.

22. The method of claim 19, wherein the C1 feedstock, the second C1 feedstock, the reverse water gas shift feedstock, or any combination thereof are pressurized prior to passing to the bioreactor.

23. The method of claim 19, wherein the fermentation product comprises at least one product selected from acetate, butyrate, 2,3-butanediol, lactate, butene, butadiene, ketones, methyl ethyl ketone, ethylene, acetone, isopropanol, lipids, 3-hydroypropionate, isoprene, fatty acids, 2-butanol, 1,2-propanediol, 1-propanol, microbial biomass, or any combination thereof.

24. The method of claim 19, wherein the reverse water gas shift process is powered, at least in part, by a renewable energy source.

25. The method of claim 24, wherein the renewable energy source is selected from solar, hydro, wind, geothermal, biomass, or combinations thereof.

26. The method of claim 19, wherein the culture further produces a post-fermentation gaseous substrate, the method further comprising passing the post-fermentation gaseous substrate to the reverse water gas shift process, the industrial or syngas process, or both.

27. The method of claim 26, wherein the post-fermentation gaseous substrate is treated to remove one or more constituents prior to being passed to the reverse water gas shift process, the industrial or syngas process, or both.

28. The method of claim 19 wherein the water electrolyzer further generates an O₂ stream that is passed to a gasifier.

29. A method of controlling a fermentation process in a bioreactor containing a bacterial culture in a liquid nutrient medium, the method comprising:
a. providing a first C1 feedstock comprising one or both of CO and CO₂ from an industrial or syngas processes, wherein the C1 feedstock has a H₂:CO₂ molar ratio and a H₂:CO molar ratio;
b. intermittently operating a reverse water gas shift process which comprises at least one reverse water gas shift reactor and a water electrolyzer by passing a second C1 feedstock comprising CO₂ from an industrial or syngas process and optionally a H₂ stream from the water electrolyzer to the reverse water gas shift reactor to produce a reverse water gas shift feedstock comprising CO, any unreacted CO₂, and any unreacted H₂, wherein the second C1 feedstock and the first C1 feedstock are the same or different, and wherein the reverse water gas shift feedstock has a H₂:CO₂ molar ratio and a H₂:CO molar ratio;
c. passing to the bioreactor
i. the first C1 feedstock when the reverse water gas shift process is not operating, or
ii. a combined feedstock comprising the C1 feedstock and the reverse water gas shift feedstock when the reverse water gas shift process is operating, the combined feed having a H₂:CO₂ molar ratio and a H₂:CO molar ratio; or
iii. either i. or ii. and the H₂ stream from the water electrolyzer;
d. fermenting, in the bioreactor, the culture using the first C1 feedstock and or the combined feedstock to produce one or more fermentation products, wherein the fermenting has a feedstock target range H₂:CO₂ molar ratio and a feedstock target range H₂:CO molar ratio; and
e. operating the reverse water gas shift process during periods of time when the H₂:CO₂ molar ratio or the H₂:CO molar ratio of the C1 feedstock is not within the fermenting feedstock target range H₂:CO₂ molar ratio or feedstock target range H₂:CO molar ratio.

30. The method of claim 29, wherein the first C1 feedstock, the second C1 feedstock, the reverse water gas shift feedstock, or any combination thereof are treated to remove one or more constituents prior to passing to the bioreactor.

31. The method of claim 29, wherein the C1 feedstock, the C2 feedstock, the reverse water gas shift feedstock, or any combination thereof are pressurized prior to passing to the bioreactor.

32. The method of claim 29, wherein the fermentation product comprises at least one product selected from acetate, butyrate, 2,3-butanediol, lactate, butene, butadiene, ketones, methyl ethyl ketone, ethylene, acetone, isopropanol, lipids, 3-hydroypropionate, isoprene, fatty acids, 2-butanol, 1,2-propanediol, 1-propanol, microbial biomass, or any combination thereof.

33. The method of claim 29, wherein the reverse water gas shift process is powered, at least in part, by a renewable energy source.

34. The method of claim 33, wherein the renewable energy source is selected from solar, hydro, wind, geothermal, biomass, or combinations thereof.

35. The method of claim 34, wherein the culture further produces a post-fermentation gaseous substrate, the method further comprising passing the post-fermentation gaseous substrate to the reverse water gas shift process, the industrial or syngas process, or both.

36. The method of claim 35, wherein the post-fermentation gaseous substrate is treated to remove one or more constituents prior to being passed to the reverse water gas shift process, the industrial or syngas process, or both.
